(19) United States
(12) Patent Application Publication
(10) Pub. No.: US 2008/0124585 A1
(43) Pub. Date: May 29, 2008

(54) Compositions and Methods for Bioelectricity Production

(75) Inventor: Christophe H. Schilling, San Diego, CA (US)

Correspondence Address: MCDERMOTT, WILL & EMERY, 4370 LA JOLLA VILLAGE DRIVE, SUITE 700, SAN DIEGO, CA, 92122, US

(73) Assignee: Genomatica, Inc., San Diego, CA (US)

(21) Appl. No.: 11/885,839

(22) PCT Filed: Mar. 10, 2006
(86) PCT No.: PCT/US06/08760

§ 371 Date: Sep. 7, 2007

Related U.S. Application Data
(60) Provisional application No. 60/660,607, filed on Mar. 10, 2005.
(60) Provisional application No. 60/689,609, filed on Jun. 9, 2005.

(51) Int. Cl. H01M 8/16; C12N 1/21

(52) U.S. Cl. 429/2; 435/252.3

(57) ABSTRACT

The invention provides a microbial fuel cell having a dissimilatory metal-reducing microbe expressing exogenous or native ATPase subunits, the ATPase subunits assembling into an active ATP synthase and consuming ATP in a futile cycle. The dissimilatory metal-reducing microbe can include an organism selected from the organisms set forth in Table 1. The one or more exogenous ATPase subunits can include a subunit selected from the ATPase subunits set forth in Tables 2 or 3. Also provided is a microbial fuel cell having a dissimilatory metal-reducing microbe expressing one or more exogenous genes encoding a gene product that promotes ATP consumption, the gene products of the one or more exogenous genes having an activity that reduces ATP synthesis, increases ATP consumption or both. The one or more gene products can increase ATP consumption through a futile cycle or through altering a metabolic reaction directly involved in ATP synthesis. Further provided is a microbial fuel cell having a dissimilatory metal-reducing microbe expressing one or more exogenous genes encoding a gene products that increases the electron/mole ratio compared to an unmodified microbe, wherein the increased ratio enhances electron transfer to an electrode. A method of producing electricity from an microbial organism is further provided. The method includes: (a) culturing a microbial fuel cell under anaerobic conditions sufficient for growth, the microbial fuel cell comprising a dissimilatory metal-reducing microbe expressing exogenous ATPase subunits, the ATPase subunits assembling into an active ATP synthase and consuming ATP in a futile cycle when grown under anaerobic conditions, and (b) capturing electrons produced by an increased ATP demand with an electron acceptor.

### BACKGROUND OF THE INVENTION

There is a pressing need to reduce our reliance on energy derived from fossil fuels, and develop alternative strategies for the generation of energy from renewable resources. One such strategy aims to directly convert carbohydrates into electrical energy by using the reducing potential inherent in biological systems whereby introducing the concept of microbially-driven fuel cells.

A microbial fuel cell is basically a system that harvests electrons produced during microbial metabolism and channels them for electric current generation. These type of fuel cells allow compounds such as simple carbohydrates or waste organic matter to be converted into electricity¹. One form of a microbial fuel cell uses artificial redox mediators that are capable of penetrating bacterial cells. When added to a culture solution within an anodic fuel cell compartment, these mediators enable electrons produced during fermentation or other metabolic processes to be shuttled to the anode. A drawback associated with these microbial fuels cells is that the microbes oxidize only a part of the substrates and also require soluble mediators to facilitate electron transfer, which can be costly. In some cases, these mediators are even toxic and cannot be used for electricity generation in open environments.

Another concept in the construction of microbial fuel cells resulted from the observation² that if graphite or platinum electrodes were placed into anoxic marine sediments, and connected to similar electrodes in the overlying oxic water, sustained electrical power could be harvested (on the order of 0.01 Watts/m² of electrode). This finding has led to the discovery that specific groups of microorganisms, most notably the Geobacteraceae, are capable of directly transferring electrons to electrodes, without the need for mediators³⁻⁵. Recently, organisms from the species Rhodoferax ferrireducens were shown to oxidize glucose to CO₂ and quantitatively transfer electrons to graphite electrodes without the need for an electron-shuttling mediator⁶. Furthermore, the recovery of electrons from glucose oxidation was over 80% of that theoretically available from glucose oxidation.

### SUMMARY OF THE INVENTION

The invention provides a microbial fuel cell having a dissimilatory metal-reducing microbe expressing exogenous or native ATPase subunits, the ATPase subunits assembling into an active ATP synthase and consuming ATP in a futile cycle. The dissimilatory metal-reducing microbe can include an organism selected from the organisms set forth in Table 1. The one or more exogenous ATPase subunits can include a subunit selected from the ATPase subunits set forth in Tables 2 or 3. Also provided is a microbial fuel cell having a dissimilatory metal-reducing microbe expressing one or more exogenous genes encoding a gene product that promotes ATP consumption, the gene products of the one or more exogenous genes having an activity that reduces ATP synthesis, increases ATP consumption or both. The one or more gene products can increase ATP consumption through a futile cycle or through altering a metabolic reaction directly involved in ATP synthesis. Further provided is a microbial fuel cell having a dissimilatory metal-reducing microbe expressing one or more exogenous genes encoding a gene products that increases the electron/mole ratio compared to an unmodified microbe, wherein the increased ratio enhances electron transfer to an electrode. A method of producing electricity from an microbial organism is further provided. The method includes: (a) culturing a microbial fuel cell under anaerobic conditions sufficient for growth, the microbial fuel cell comprising a dissimilatory metal-reducing microbe expressing exogenous ATPase subunits, the ATPase subunits assembling into an active ATP synthase and consuming ATP in a futile cycle when grown under anaerobic conditions, and (b) capturing electrons produced by an increased ATP demand with an electron acceptor.

### BRIEF DESCRIPTION OF THE DRAWINGS

FIG. 1 shows a genome based in silico model. The analysis of the metabolic network of G. sulfurreducens using the Simpheny™ platform allowed identification of potential substrates with high electron/mol ratio. The predicted flux for acetate was 71 mmol/10 mM acetate. The predicted flux for glycerol was 65 mmol/10 mM glycerol.

FIG. 2 shows the effect on bioelectricity production when alternate substrates are utilized. The glycerol processing operon from D. vulgaris was cloned in pRG plasmid (Dglyop) and expressed in G. sulfurreducens. The engineered strain containing the glycerol transporter was able to grow in NBAF with glycerol (Gly) while the wild type was comparable to growth with a minimum concentration of Acetate (Ac). The engineered strain also was able to grow with Glycerol as the only carbon and electron source on iron oxide.

FIG. 3 shows the effect on bioelectricity production when respiration rate is increased. The left panel shows growth of the modified ATPase expressing G. sulfurreducens strain under increasing ATPase induction. The right panel shows the level of respiration in the presence or absence of increased respiration caused by ATPase induction.

FIG. 4 shows bioelectricity production and direct transfer of electrons to an electrode using engineered Geobacter cells. A two-chambered microbial fuel cell is shown in the left panel of FIG. 4. The right panel of FIG. 4 shows current generation following ATPase induction.

### DETAILED DESCRIPTION OF THE INVENTION

Direct transfer of electrons to electrodes can be harnessed for the production of electricity by biological organisms. For example, microbial cells can be attached to electrodes as catalysts for harvesting electricity from sources such as organic wastes, carbohydrate, feedstocks, and contaminated groundwaters. Thus, in this alternative form of a fuel cell, metal-reducing bacteria are incorporated that partly exhibit special membrane-bound cytochromes capable of transferring electrons directly to the electrodes rather than having to use a redox mediator to shuttle electrons to the anode. Bioelectricity production can be augmented to increase amounts sufficient for commercial purposes employing the genetic modifications described below. Further, because the bioelectrical enhancements described herein rest on genetic compositions and gene product expression levels or activity, the bioelectrical organisms of the invention can be genetically modified to modulate the expression or activity of one, some or all of the molecular components of the bioelectricity machinery in order to increase or decrease bioelectricity production.

The invention is directed to the metabolic engineering of dissimilatory metal reducing microbes so as to channel more electrons through the respiratory machinery of a cell for transfer to an electrode. Increasing respiratory electron flow can be accomplished by, for example, increasing the ATP/energy demand that is placed on the cells whereby forcing the cells to generate more ATP. Increasing ATP production will in turn increase bioelectricity production by transferring more electrons to an external electrode.

Bioelectricity production can be generated in a variety of organisms. A particularly useful organism is Geobacter sulfurreducens. However, metabolic engineering to increase ATP production with a concomitant increase in electron transfer and electrical production is applicable, for example, to all dissimilatory metal-reducing microbe for use in a microbial fuel cell. G. sulfurreducens, is a particular member of the class of dissimilatory metal reducing bacteria, with applications in bioremediation and bioelectricity generation. This microorganism belongs to the Geobacteraceae family, that have been shown to be a dominant member of the communities of bacteria associated with uranium bioremediation^{7,8}, and in bioelectricity generation in microbial fuel cells.

Previous rates of transfer of electrons in G. sulfurreducens is quite slow and can support, if at all, only very low powered devices. Hence, there is a critical need to genetically engineer the metabolism of these and other organisms to enhance the rate of electron transport, so that these microbial fuel cells become commercially practical.

The application of metabolic engineering has been used to synthesize bulk commodity chemicals such as 1,3 propanediol, acetate, lactate, and other metabolites.

The invention is directed to the engineering of microorganisms to enhance the rate of electron transfer to electrodes, through the introduction of heterologous genes into the genome of such microorganisms. For example, G. sulfurreducens, for which current rates of electron transfer are low and a genetic system has been identified to facilitate the insertion of novel genes⁹, can be engineered to increase bioelectrical production over previously obtained electron transfer rates. By modulating heterologous gene expression substantial increases can be observed over that previously obtained.

As described previously, initial metabolic engineering attempts have primarily focused on increasing the supply of metabolic enzymes. However, merely increasing the supply of metabolic enzymes in a pathway often fails to increase the product synthesis rate, as the interactions between the different subsets of metabolism are not considered in this simple strategy. Recently, metabolic engineering through demand management has been proposed¹⁰, where the demand of key intermediates such as ATP is engineered. This concept has been attempted for increasing the flux through the glycolytic enzymes¹¹ and for the production of acetate¹² in Escherichia coli. However, engineering of important intermediates has never been contemplated for enhancing the transfer of electrons to an electrode for electricity generation.

In the first instance described above, where the glycolytic flux was desired to be increased, an ATPase consisting of the genes encoding the alpha, beta, and the gamma subunits of the ATP synthase was introduced into E. coli. These subunits of the ATP synthase act as a cytoplasmic ATPase. The ATPase created a futile cycle that increased ATP consumption and increased the glycolytic flux as the demand for ATP increased. In the second instance described above, the genes corresponding to the F₀ part of the (F₁F₀)H⁺ ATP synthase was deleted, creating a cytoplasmic ATPase that lead to a futile cycle consuming ATP. Since, the only fermentation pathway available was the acetate production pathway that regenerated ATP, the acetate production of up to 75% of the maximum theoretical yield was obtained.

In Geobacter sulfurreducens, the rate of electron transfer through the electron transport chain depends on the efficiency of the chain. For example, for growth on Fe(III), the yield on acetate is three times lower than for growth on fumarate, and the rates of electron transport is higher for growth on Fe(III).

The invention provides organisms having a gene operatively inserted for an ATPase that when expressed will cause consumption of ATP. This metabolic result in turn will increase the demand for the production of ATP by the cell's metabolic machinery. In dissimilatory metal-reducing microbes this increased demand can be met, for example, by channeling more protons out of the cell to produce more ATP via the proton-gradient. This result comes with the concomitant channeling of more electrons through the respiratory chain ending with the transfer of these electrons to an electron acceptor such as a graphite electrode.

An alternative possibility is to decrease the efficiency of the electron transport chain, so that more electrons flow through the chain to generate equivalent amounts of ATP. In both of the above bioelectricity modes, the activity of the operatively inserted ATPase and the degree of the efficiency can be controlled so that the cell maintains homeostasis. In this regard, controlling the efficiency ensures that the cell is not overwhelmed by the increased energy demand as these organisms could be potentially energetically limited for growth. The inserted ATPase genes can be placed, for example, under the control of a promoter so that the expression of the ATPase can be initiated once there is sufficient build-up of the organism's biomass.

The invention also provides a microbial fuel cell having a dissimilatory metal-reducing microbe expressing one or more exogenous or native genes encoding a gene products that promote ATP consumption, the gene products of the one or more exogenous or native genes having an activity that reduces ATP synthesis, increases ATP consumption or both.

The invention has been exemplified by reference to an embodiment that causes ATP consumption through the expression of an ATPase. Given the teachings and guidance provided herein, those skilled in the art will understand that essentially any gene or gene modification that promotes ATP consumption will similarly increase the demand for ATP production and concomitant increase of electron flux through the respiratory chain. This result can be accomplished by, for example, genetically modifying a microbe to increases ATP consumption through a futile cycle resulting in reduced ATP synthesis and/or increased ATP consumption.

The genetic modifications can include metabolic reactions or pathways directly involved in ATP synthesis. Such modifications include, for example, inactivating an ATP synthesis gene. Inactivation can be accomplished by, for example, introducing one or more mutations, substituting one or more genes with a non-functional exogenous gene by site specific recombination or by expressing a regulator or inhibitor of a gene directly involved in ATP synthesis. Specific examples of such gene products include the genes for phosphofructokinase, and pyruvate kinase. By coupling the expression of phosphofructokinase with a fructose-bisphoshotase, a futile cycle that dissipates ATP can increase the consumption of ATP. Similarly a futile cycle can be created by simultaneous use of pyruvate kinase and phosphoenolpyruvate synthase, or any kinase enzyme and it's reciprocal phosphatase enzyme.

Alternatively, ATP consumption can be accomplished by, for example, genetic modifications of metabolic reactions or pathways indirectly involved in ATP synthesis. Genes indirectly involved in ATP synthesis include gene products that act a distal point such as at a precursor pathway or it blocks the coupling of ATP synthesis to electron transport. Such modifications include, for example, introducing one or more mutations, substituting one or more genes with a non-functional exogenous gene by site specific recombination or by expressing a regulator or inhibitory of a gene indirectly involved in ATP synthesis.

The above described metabolic engineering for bioelectricity production also can be applied, for example, to any organism, natural or engineered, that transfers electrons to an electrode, to enhance the generation rate of electrical current. The operable introduction of ATPases also can be successfully applied to all dissimilatory metal reducing microbes where, for example, the metal reduction is coupled to growth or coupled to other microbes, including fermentative or sulfate reducing microbes such as Clostridium beijerinkii¹³ or Desulfotomaculum reducens¹⁴, where the metal reduction can be coupled to growth, for example. Exemplary dissimilatory metal reducing microbes that can be metabolically engineered to produce practical quantities of bioelectricity are set forth below in Table 1.

TABLE 1
Exemplary Dissimilatory Metal Reducing Microbes

| Kingdom | Intermediate Rank | Genus | Species |
|---|---|---|---|
| Bacteria | delta subdivision | Geobacter | bremensis, chapelleii, |
| | proteobacteria | | grbiciae, |
| | | | hydrogenophilus, |
| | | | metallireducens, |
| | | | pelophilus, |
| | | | sulfurreducens |
| Bacteria | delta subdivision | Geothermobacter | ehrlichii |
| | proteobacteria | | |
| Bacteria | Acidobacteria | Geothrix | fermentans |
| Bacteria | beta subdivision | Rhodoferax | ferrireducens |
| | proteobacteria | | |
| Bacteria | gamma subdivision | Shewanella | amazonensis, |
| | proteobacteria | | frigidimarina, |
| | | | gelidimarina, |
| | | | oneidensis, olleyana, |
| | | | livingstonensis |
| Bacteria | Thermodesulfobacteria | Geothermobacterium | ferrireducens |
| Bacteria | Thermotogae | Thermotoga | maritima |
| Archae | Thermoprotei crearchaeota | Pyrobaculum | islandicum |
| Archae | Arcaeoglobi euryachaeota | Geoglobus | ahangari |

For the production of bioelectricity producing microbes, genes encoding an ATPase can be introduced in operable form for expression and functional assembly of the encoded gene products. Briefly, the genes encoding the F₁ part of the ATPase can come from essentially any organism including, for example, any of the several organisms shown below in Table 2. The genes coding for the corresponding subunits in eukaryotic species such as Saccharomyces cerevisiae can also be incorporated into the dissmilatory metal reducing bacteria. In these cases, codon optimization to eliminate rare codons in the eukaryotic genes could be necessary to increase the expression of the gene products.

### In addition to the F-type ATPase, the genes coding for the V₁ subunit of the V type ATPase shown in Table 3 or the A-type ATPase¹⁵ can also be inserted into the dissimilatory metal reducing bacteria for creating an extra ATP demand.

In the specific instance of Geobacter sulfurreducens, the gene coding for the F₁ part of the ATPase from, for example, Escherichia coli can be introduced into a microbe of the invention and expressed for bioelectricity production. An exemplary vector useful for introduction and expression is the plasmid pCM66, a high copy-number plasmid that is stable in G. sulfurreducens even in the absence of antibiotic pressure. The genes coding for the F₁ATPase (atpAGD coding for the alpha, beta, gamma subunits in E. coli) can be, for example, cloned into this plasmid under the control of either a constitutive or inducible promoter. Constitutive promoters can be chosen that exhibit different expression strengths to achieve a desired level of exogenous ATPase expression. These genes can be obtained from the source organism or organisms or from source plasmids using restriction enzymes followed by amplification with sequence specific primers or other recombinant techniques well known to those skilled in the art. The gene can then be cloned into the host plasmid and the cells cultured for polypeptide expression and self-assembly of the ATPase subunits. The expression of these genes can be verified by subsequent analysis including, for example, RNA expression, polypeptide expression or activity measurements. These analysis as well as other means for determining the level or activity of an exogenously expressed polypeptide are well known to those skilled in the art.

In addition, all of the above designs and methods for expressing ATPase encoding nucleic acids for the consumption of ATP also can be applied to the expression of non-ATPase genes or metabolic regulators, for example, that similarly increase the consumption of ATP which can be harnessed for the production of bioelectricity. For example, a futile cycle can be created by coordinated expression of genes for phosphofructokinase and fructose-bisphosphotase that will result in a net reaction that consumes ATP.

The invention also provides a microbial fuel cell having a dissimilatory metal-reducing microbe expressing one or more exogenous genes encoding a gene products that increases the electron/mole ratio compared to an unmodified microbe, wherein the increased ratio enhances electron transfer to an electrode. A specific example of altering the carbon or substrate utilization to increase electron transfer is described further below in Example I where the one or more gene products confers glycerol processing activity. Other carbon or substrate utilization sources that can provide increased electron/mole ratio are well known in the art. These include carbohydrates such as glucose, fructose, arabinose, and xylose, as well as benzene.

Once the foreign genes are expressed in the host organism in a stable manner, consisting of, for example, two or more generations, the bioelectricity producing strains can be evaluated for enhanced electricity production in, for example, an electrode-containing chamber. Briefly, G. sulfurreducens can be grown in temperature-controlled, anaerobic, two-chambered electrode cells, under control of a potentiostat. The more tightly regulated the anaerobic conditions can be maintained, the greater the ATP consumption and the more efficient production of bioelectricity can be achieved. A graphite electrode can be poised at a fixed potential and serves as a consistent electron acceptor for the dissimilatory metal reducing bacteria. Output from multiple potentiostats can be continuously logged via a computerized data logging system, allowing multiple strains or conditions to be assessed simultaneously.

Using this system, for example, the rate of electron transport to electrodes can be directly measured under controlled conditions, and following measurement of the amount of biomass attached to electrodes, the rates can be expressed per unit cell mass for comparisons. To examine the abilities of unengineered and engineered strains, cells can be grown on electrodes using similar concentrations of a common electron donor, such as acetate. Following this establishment phase, for example, the medium surrounding the electrodes can be removed and replaced with fresh, anaerobic medium. The biofilms which remain attached to the electrodes can be measured, for example, for their ability to transfer electrons and the rate of electrical current generation could be measured to demonstrate the improved power generation capabilities. The improvement in the electrical current generation will enable the creation of microbial fuel cells that can generate higher power, thereby making the biological fuel cells of the invention commercially viable.

TABLE 2
Representative orthologs coding for the alpha, beta, and gamma subunits of the F₁ ATPase

| | alpha subunit | | beta subunit | | gamma subunit | |
|---|---|---|---|---|---|---|
| Organism | Locus | Gene | Locus | Gene | Locus | Gene |
| Agrobacterium tumefaciens C58 | Atu2624 | atpA | Atu2622 | atpD | Atu2623 | atpG |
| Anabaena sp. PCC7120 (Nostoc sp. PCC7120) | all0005 | atpA | all5039 | atpB | all0004 | atpC |
| Aquifex aeolicus | aq_679 | atpA | aq_2038 | atpD | aq_203 | atpG2 |
| Bacillus anthracis | BA5549 | atpA | BA5547 | atpD | BA5548 | atpG |
| Bacillus halodurans | BH3756 | atpA | BH3754 | atpD | BH3755 | atpG |
| Bacillus subtilis | BG10819 | atpA | BG10821 | atpD | BG10820 | atpG |
| Bifidobacterium longum | BL0359 | atpA | BL0357 | atpD | BL0358 | atpG |
| Blochmannia floridanus | Bfl006 | atpA | Bfl008 | atpD | Bfl007 | atpG |
| Bordetella bronchiseptica | BB4607 | atpA | BB4605 | atpD | BB4606 | atpG |
| Bordetella parapertussis | BPP4137 | atpA | BPP4135 | atpD | BPP4136 | atpG |
| Bordetella pertussis | BP3286 | atpA | BP3288 | atpD | BP3287 | atpG |
| Bradyrhizobium japonicum | bll0442 | atpA | bll0440 | atpD | bll0441 | atpG |
| Brucella suis | BR1801 | atpA | BR1799 | atpD | BR1800 | atpG |
| Buchnera aphidicola Bp | bbp006 | atpA | bbp008 | atpD | bbp007 | atpG |
| Buchnera aphidicola Sg | BUsg006 | atpA | BUsg008 | atpD | BUsg007 | atpG |
| Buchnera sp. APS | BU006 | atpA | BU008 | atpD | BU007 | atpG |
| Campylobacter jejuni | Cj0105 | atpA | Cj0107 | atpD | Cj0106 | atpG |
| Chromobacterium violaceum | CV0670 | atpA | CV0672 | atpD | CV0671 | atpG |
| Clostridium acetobutylicum | CAC2867 | atpA | CAC2865 | atpD | CAC2866 | atpG |
| Clostridium perfringens | CPE2189 | atpA | CPE2187 | atpB | CPE2188 | atpG |
| Corynebacterium diphtheriae | DIP1050 | atpA | DIP1052 | atpD | DIP1051 | atpG |
| Corynebacterium efficiens | CE1313 | atpA | CE1315 | atpB | CE1314 | atpG |
| Coxiella burnetii | CBU1943 | atpA | CBU1945 | atpD | CBU1944 | atpG |
| Enterococcus faecalis | EF2610 | atpA | EF2608 | atpD | EF2609 | atpG |
| Escherichia coli CFT073 | c4660 | atpA | c4658 | atpD | c4659 | atpG |
| Escherichia coli K-12 MG1655 | b3734 | atpA | b3732 | atpD | b3733 | atpG |
| Escherichia coli K-12 W3110 | JW3712 | atpA | JW3710 | atpD | JW3711 | atpG |
| Escherichia coli O157 EDL933 | Z5232 | atpA | Z5230 | atpD | Z5231 | atpG |
| Geobacter sulfurreducens | GSU0111 | atpA | GSU0113 | atpD | GSU0112 | atpG |
| Gloeobacter violaceus | gll2905 | atpA | gll2570 | atpB | glr4315 | atpC |
| Haemophilus ducreyi | HD0008 | atpA | HD0010 | atpD | HD0009 | atpG |
| Haemophilus influenzae | HI0481 | atpA | HI0479 | atpD | HI0480 | atpG |
| Helicobacter hepaticus | HH0427 | atpA | HH0429 | atpD | HH0428 | atpG |
| Helicobacter pylori 26695 | HP1134 | atpA | HP1132 | atpD | HP1133 | atpG |
| Lactobacillus plantarum | lp_2366 | atpA | lp_2364 | atpD | lp_2365 | atpG |
| Lactococcus lactis | L8990 | atpA | L6563 | atpD | L8105 | atpG |
| Leptospira interrogans | LA2779 | atpA | LA2776 | atpD | LA2778 | atpG |
| Mycobacterium bovis | Mb1340 | atpA | Mb1342 | atpD | Mb1341 | atpG |
| Mycobacterium leprae | ML1143 | atpA | ML1145 | atpD | ML1144 | atpG |
| Mycobacterium tuberculosis H37Rv | Rv1308 | atpA | Rv1310 | atpD | Rv1309 | atpG |
| Mycoplasma genitalium | MG401 | atpA | MG399 | atpD | MG400 | atpG |
| Mycoplasma penetrans | MYPE600 | atpA | MYPE620 | atpD | MYPE610 | atpG |
| Mycoplasma pneumoniae | D02_orf518 | atpA | D02_orf475 | atpD | D02_orf279 | atpG |
| Neisseria meningitidis Z2491 | NMA0517 | atpA | NMA0519 | atpD | NMA0518 | atpG |
| Nitrosomonas europaea | NE0204 | atpA | NE0206 | atpD | NE0205 | atpG |
| Oceanobacillus iheyensis | OB2977 | atpA | OB2975 | atpD | OB2976 | atpG |
| Pasteurella multocida | PM1492 | atpA | PM1494 | atpD | PM1493 | atpG |
| Photorhabdus luminescens | plu0042 | atpA | plu0040 | atpD | plu0041 | atpG |
| Prochlorococcus marinus MED4 | PMM1451 | atpA | PMM1438 | atpB | PMM1450 | atpC |
| Prochlorococcus marinus MIT9313 | PMT1467 | atpA | PMT1451 | atpB | PMT1466 | atpC |
| Prochlorococcus marinus SS120 | Pro1604 | atpA | Pro1591 | atpD | Pro1603 | atpG |
| Pseudomonas aeruginosa | PA5556 | at | PA5554 | atpD | PA5555 | atpG |
| Pseudomonas putida | PP5415 | atpA | PP5413 | atpD | PP5414 | atpG |
| Pseudomonas syringae pv. tomato | PSPTO5601 | atpA | PSPTO5599 | atpD | PSPTO5600 | atpG |
| Ralstonia solanacearum | RS02549 | atpA | RS02547 | atpD | RS02548 | atpG |
| Rhodopsdudomonas palustris | RPA0178 | atpA | RPA0176 | atpD | RPA0177 | atpG |
| Rickettsia conorii | RC1237 | atpA | RC1235 | atpD | RC1236 | atpG |
| Rickettsia prowazekii | RP803 | atpA | RP801 | atpD | RP802 | atpG |
| Salmonella typhi CT18 | STY3911 | atpA | STY3913 | atpD | STY3912 | atpG |
| Salmonella typhi Ty2 | t3652 | atpA | t3654 | atpD | t3653 | atpG |
| Salmonella typhimurium | STM3867 | atpA | STM3865 | atpD | STM3866 | atpG |
| Shewanella oneidensis | SO4749 | atpA | SO4747 | atpD | SO4748 | atpG |
| Shigella flexneri 2457T | S3954 | atpA | S3956 | atpD | S3955 | atpG |
| Shigella flexneri 301 | SF3814 | atpA | SF3812 | atpD | SF3813 | atpG |
| Sinorhizobium meliloti | SMc02499 | atpA | SMc02501 | atpD | SMc02500 | atpG |
| Staphylococcus aureus Mu50 (VRSA) | SAV2105 | atpA | SAV2103 | atpD | SAV2104 | atpG |
| Staphylococcus aureus MW2 | MW2029 | atpA | MW2027 | atpD | MW2028 | atpG |
| Staphylococcus aureus N315 (MRSA) | SA1907 | atpA | SA1905 | atpD | SA1906 | atpG |
| Streptococcus agalactiae 2603 | SAG0861 | atpA | SAG0863 | atpD | SAG0862 | atpG |
| Streptococcus agalactiae NEM316 | gbs0879 | atpA | gbs0881 | atpD | gbs0880 | atpG |
| Streptococcus mutans | SMU.1530 | atpD | SMU.1528 | atpB | SMU.1529 | atpC |
| Streptococcus pneumoniae R6 | spr1362 | atpA | spr1360 | atpD | spr1361 | atpG |
| Streptococcus pyogenes MGAS8232 | spyM18_0816 | atpA | spyM18_0818 | atpD | spyM18_0817 | atpG |
| Streptococcus pyogenes SF370 | SPy0758 | atpA | SPy0760 | atpD | SPy0759 | atpG |
| Streptomyces avermitilis | SAV2883 | atpA | SAV2881 | atpD | SAV2882 | atpG |
| Streptomyces coelicolor | SCO5371 | 2SC6G5.15 | SCO5373 | 2SC6G5.17 | SCO5372 | 2SC6G5.16 |
| Synechococcus sp. WH8102 | SYNW0494 | atpA | SYNW0512 | atpB | SYNW0495 | atpC |
| Synechocystis sp. PCC6803 | sll1326 | atpA | slr1329 | atpB | sll1327 | atpC |
| Thermoanaerobacter tengcongensis | TTE0635 | atpA | TTE0637 | atpD | TTE0636 | atpG |
| Thermosynechococcus elongatus | tlr0435 | atpA | tlr0525 | atpB | tll0385 | atpC |
| Tropheryma whipplei TW08/27 | TW342 | atpA | TW344 | atpD | TW343 | atpG |
| Tropheryma whipplei Twist | TW426 | atpA | TW424 | atpD | TW425 | atpG |
| Wigglesworthia brevipalpis | Wbr0132 | atpA | Wbr0130 | atpD | Wbr0131 | atpG |
| Wolinella succinogenes | WS0514 | atpA | WS0516 | atpD | WS0515 | atpG |
| Xanthomonas axonopodis | XAC3651 | atpA | XAC3649 | atpD | XAC3650 | atpG |
| Xanthomonas campestris | XCC0552 | atpA | XCC0554 | atpD | XCC0553 | atpG |
| Xylella fastidiosa Temeculal | PD0430 | atpA | PD0428 | atpD | PD0429 | atpG |
| Yersinia pestis CO92 | YPO4123 | atpA | YPO4121 | atpD | YP04122 | atpG |
| Yersinia pestis KIM | y4137 | atpA | y4135 | atpD | y4136 | atpG |
| Saccharomyces cerevisiae | YBL099W | atp1 | YJR121W | atp2 | YBR039W | atp3 |

TABLE 3
Representative orthologs coding for the A, B, and D subunits of the V₁ ATPase¹⁶

| | A subunit | | B subunit | | D subunit | |
|---|---|---|---|---|---|---|
| Organism Name | Locus | Gene | Locus | Gene | Locus | Gene |
| Archaeoglobus fulgidus | AF1166 | atpA | AF1167 | atpB | AF1168 | atpD |
| Borrelia burgdorferi | BB0094 | atpA | BB0093 | atpB | BB0092 | atpD |
| Chlamydia trachomatis | CT308 | atpA | CT307 | atpB | CT306 | atpD |
| Clostridium perfringens | CPE1638 | ntpA | CPE1637 | ntpB | CPE1636 | ntpD |
| Halobacterium sp. NRC-1 | VNG2139G | atpA | VNG2138G | atpB | VNG2135G | atpD |
| Methanococcus jannaschii | MJ0217 | atpA | MJ0216 | atpB | MJ0615 | atpD |
| Methanopyrus kandleri | MK1017 | ntpA | MK1673 | ntpB | MK1674 | ntpD |
| Methanosarcina acetivorans | MA4158 | atpA | MA4159 | atpB | MA4160 | atpD |
| Porphyromonas gingivalis | PG1803 | atpA | PG1804 | atpB | PG1805 | atpD |
| Pyrobaculum aerophilum | PAE0663 | atpA | PAE1146 | atpB | PAE0758 | atpD |
| Pyrococcus abyssi | PAB2378 | atpA | PAB1186 | atpB | PAB2379 | atpD |
| Streptococcus pyogenes MGAS315 (serotype M3) | SpyM3_0120 | ntpA | SpyM3_0121 | ntpB | SpyM3_0122 | ntpD |
| Streptococcus pyogenes SF370 (serotype M1) | SPy0154 | ntpA | SPy0155 | ntpB | SPy0157 | ntpD |
| Sulfolobus solfataricus | SSO0563 | atpA | SSO0564 | atpB | SSO0566 | atpD |

Throughout this application various publications have been referenced within parentheses. The disclosures of these publications in their entireties are hereby incorporated by reference in this application in order to more fully describe the state of the art to which this invention pertains.

It is understood that modifications which do not substantially affect the activity of the various embodiments of this invention are also included within the definition of the invention provided herein. Those skilled in the art will readily appreciate that the specific examples and studies detailed above are only illustrative of the invention. Accordingly, specific examples disclosed herein are intended to illustrate but not limit the present invention. It also should be understood that, although the invention has been described with reference to the disclosed embodiments, various modifications can be made without departing from the spirit of the invention. Accordingly, the invention is limited only by the following claims.

### EXAMPLE I

### Engineering Geobacter sulfurreducens for Enhanced Electricity Production

Previous studies have reported that Geobacteraceae can harvest electricity from waste organic matter by oxidizing organic compounds to carbon dioxide coupled to electron transfer onto electrode surfaces. Although the conversion of organic matter to electricity in this manner can be efficient, the process is slow. Furthermore, Geobacter species have a selective number of electron donors they can utilize and thus fermentative organisms are required in order to convert complex organic substrates to the organic acids that Geobacter species can oxidize. This Example describes the engineered expansion of Geobacter species substrate range to accelerate their rate of electron transfer in order to enhance electricity production.

The developmental design for engineered expansion of substrate range employed a genome-based in silico model of the physiology of Geobacter sulfurreducens. For example, glycerol has a relatively high electron per mole ratio, and the model predicted that glycerol could be used as an electron donor if the appropriate transporter was present. This prediction was confirmed by cloning the glycerol uptake and processing operon from Desulfovibrio vulgaris, another δ-proteobacterium. As predicted by the in silico model, the engineered strain of G. sulfurreducens had the ability to grow with glycerol as the sole electron donor. Furthermore, a hierarchical optimization strategy was used to identify specific in silico gene deletions that could enhance the rate of electron transport during growth on glycerol or acetate. The in silico prediction that deletions in ATP synthesizing reactions will lead to increased activity of the ATP synthase and an enhanced rate of electron transfer was confirmed. These studies further corroborate bioelectricity using the engineered organisms and methods of the invention and also demonstrate that genome-based in silico modeling of microbial physiology can significantly augment the design and implementation process for bioelectricity improvement and optimization.

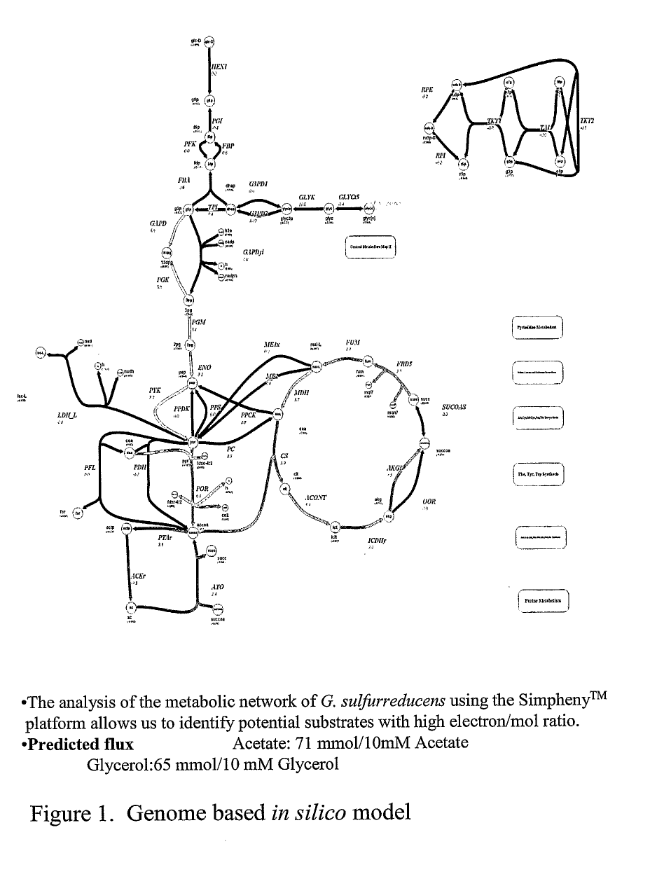

Briefly, generation and analysis of an in silico metabolic network of G. sulfurreducens was performed using the system and methods described in U.S. patent application Ser. No. 10/173,547, filed Jun. 14, 2002, entitled Systems and Methods for Constructing Genomic-Based Phenotypic Models, which is incorporated herein by reference in its entirety. These in silico systems and methods allow for the identification of potential substrates having a high electron/mole ration. As shown in FIG. 1, G. sulfurreducens was predicted to have a flux on acetate of 71 mmol/10 mM acetate. When grown on glycerol, the in silico G. sulfurreducens model also predicted a flux of 65 mmol/10 mM glycerol.

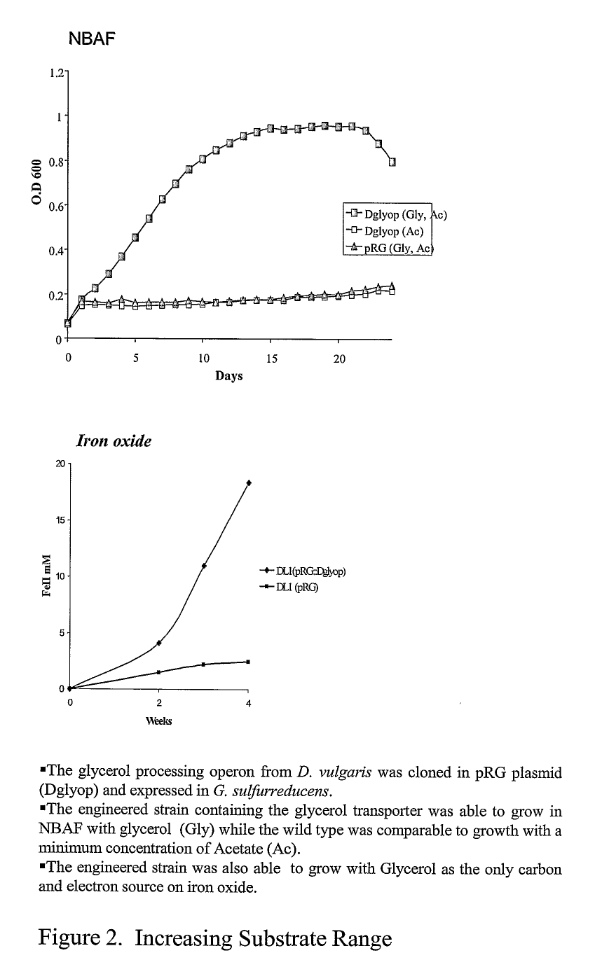

A modified G. sulfurreducens was constructed to enable it to utilize the alternative substrate glycerol by recombinantly incorporating genes encoding glycerol processing functions operably linked for expression. In this regard, a glycerol processing operon from D. vulgaris was cloned in pRG plasmid (Dglyop) and expressed in G. sulfurreducens using methods well known to those skilled in the art. As shown in FIG. 2 (top), the engineered strain containing the glycerol transporter was able to grow in NBAF with glycerol (Gly) while the wild type was comparable to growth with a minimum concentration of Acetate (Ac). The engineered strain also was able to grow with glycerol as the only carbon and electron source on iron oxide (FIG. 2 (bottom)).

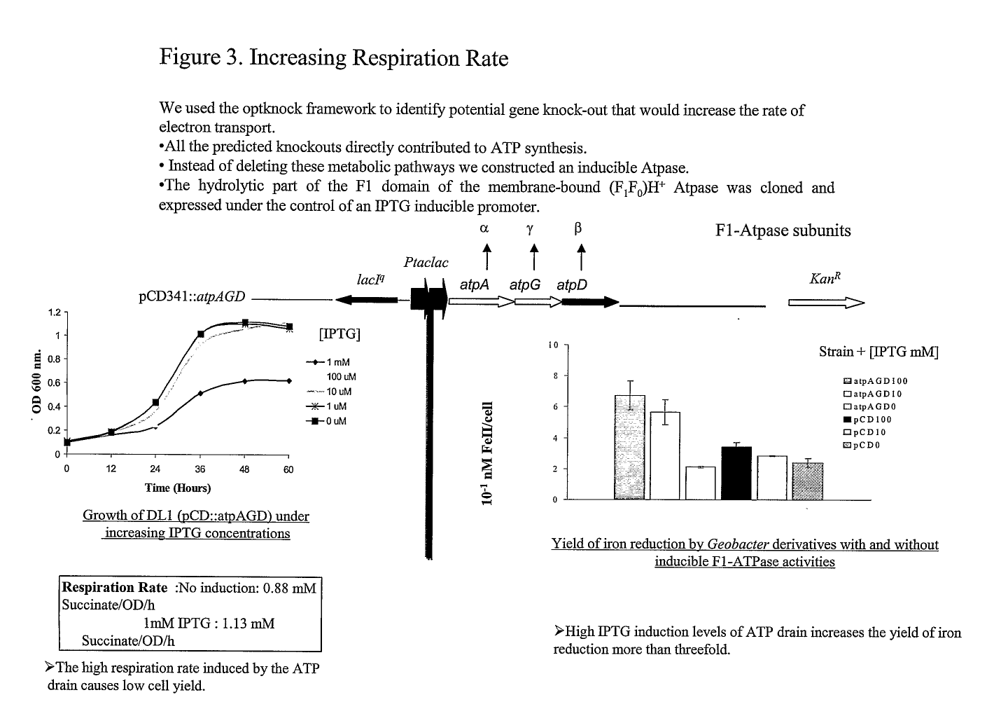

The modified glycerol-utilizing G. sulfurreducens strain was engineered to increase the respiration rate for efficient bioelectricity production. Briefly, the optknock framework of the in silico strain was used to identify potential gene knock-out that would increase the rate of electron transport. All predicted knockouts were identified as directly contributing to ATP synthesis. One means of increasing the respiration rate can be by deleting one or more of the identified genes. Alternatively, the modified glycerol-utilizing G. sulfurreducens strain was engineered to contain an inducible ATPase. To do this, the hydrolytic portion of the F₁ domain of the membrane-bound (F₁F₀)H⁺ ATPase was cloned and expressed under the control of an IPTG inducible promoter. The inducible promoter utilized was the lac Z promoter and the ATPase subunits α, β and γ were expressing as an operon as illustrated in the construct shown in FIG. 3. Further, the left panel shows growth of the modified ATPase expressing G. sulfurreducens strain under increasing ATPase induction. The right panel shows the level of respiration in the presence or absence of increased respiration caused by ATPase induction. The results indicate that high respiration rate induced by the ATP drain reduced cell yield. However, high IPTG induction levels of ATP consumption also increased the yield of iron reduction by more than threefold.

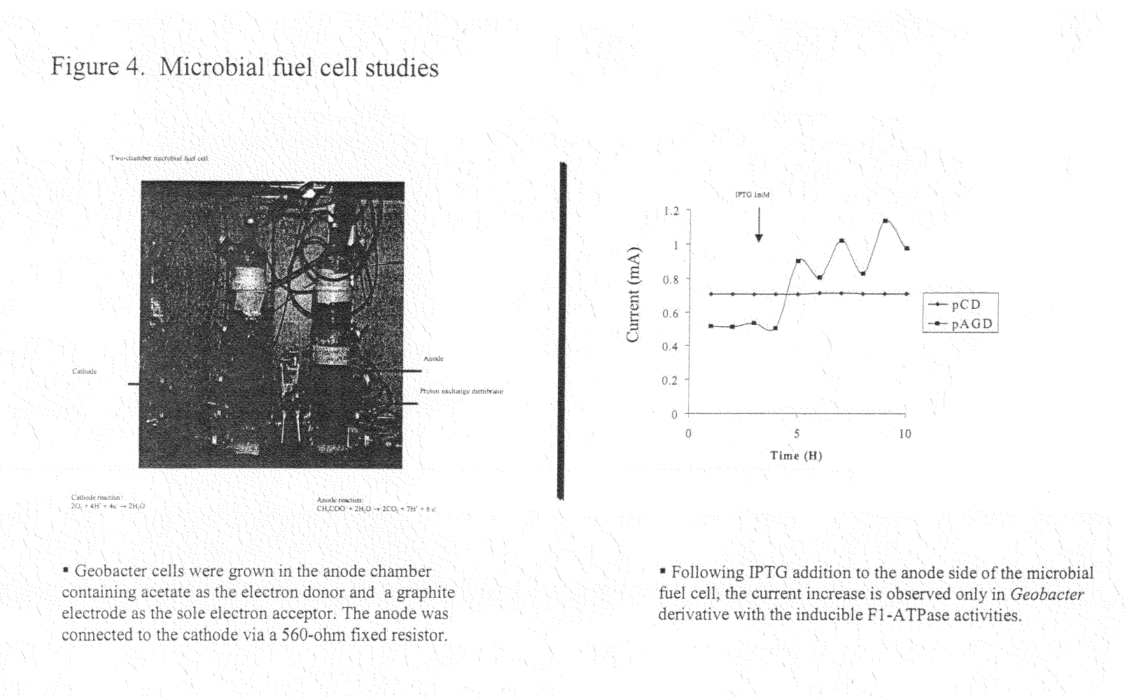

To demonstrate the ability of the modified glycerol-utilizing G. sulfurreducens strains expressing ATPase can generate electricity and directly transfer of electrons to an electrode, these engineered Geobacter cells were grown in an anode chamber containing acetate as the electron donor and a graphite electrode as the electron acceptor. The anode was connected to the cathode via a 560-ohm fixed resistor. This two-chambered microbial fuel cell is shown in the left panel of FIG. 4. The right panel of FIG. 4 shows current generation following ATPase induction. The results indicate that following IPTG addition to the anode side of the microbial fuel cell, the current increase is observed only in the engineered Geobacter strain having an inducible F1-ATPase activities. These results corroborate that bioelectricity can be produced by modifying a cell or organism to increase ATP consumption. These results further exemplify that numerous genetic designs other than ATPase expression can be implemented to increase the level of ATP consumption for enhanced production of bioelectricity. Identification, design and implementation can be particularly efficient using in silico models to identified reactions and pathways that can be modified to confer physiological properties beneficial to enhancing ATP consumption. Thus, the results further confirm that microbial fuel cells converting renewable biomass to electricity can be generated with high efficiency.

### REFERENCES

1. Wingard et al., Enzyme and Microbial Technology 4, 137-142 (1982).
2. Reimers et al., Environmental Science & Technology 35, 192-195 (2001).
3. Bond et al., Science 295, 483-485 (2002).
4. Tender et al., Nat. Biotechnol. 20, 821-825 (2002).
5. Holmes, D. E. & Bond, D. R. Microbial communities associated with electron-accepting and electron-donating electrodes in sediment fuel cells. Submitted (2003).
6. Chaudhuri et al., Nat. Biotechnol. 21, 1229-1232 (2003).
7. Holmes et al., Appl. Environ. Microbiol. 68, 2300-2306 (2002).
8. Cervantes et al., Biotechnology Letters 25, 39-45 (2003).
9. Coppi et al., Appl Environ Microbiol 67, 3180-3187 (2001).
10. Oliver, S., Nature 418, 33-34 (2002).
11. Koebmann et al., Journal of Bacteriology 184, 3909-3916 (2002).
12. Causey et al., Proc. Natl. Acad. Sci. USA 100, 825-832 (2003).
13. Dobbin et al., FEMS Microbiol. Lett. 176, 131-138 (1999).
14. Tebo et al., Fems Microbiology Letters 162, 193-198 (1998).
15. Gruber et al., J. Exp. Biol. 204, 2597-2605 (2001).
16. Nishi et al., Nat. Rev. Mol. Cell. Biol. 3, 94-103 (2002).

What is claimed is:

1. A microbial fuel cell, comprising a dissimilatory metal-reducing microbe expressing exogenous or native ATPase subunits, said ATPase subunits assembling into an active ATP synthase and consuming ATP in a futile cycle.

2. The microbial fuel cell of claim 1, wherein said dissimilatory metal-reducing microbe comprises an organism selected from the organisms set forth in Table 1.

3. The microbial fuel cell of claim 1, wherein one or more exogenous ATPase subunits comprise a subunit selected from the ATPase subunits set forth in Tables 2 or 3.

4. The microbial fuel cell of claim 1, wherein said futile cycle of ATP consumption produces a flow of electrons through the respiratory machinery at a rate higher than endogenous electron flow of about 2-fold, preferable about 5-fold, more preferably about 10-fold or more.

5. The microbial fuel cell of claim 1, wherein said futile cycle of ATP consumption produces a flow of electrons through the respiratory machinery at a rate higher than endogenous electron flow of about 20-fold, preferably about 25-fold, more preferably about 50-fold or more.

6. The microbial fuel cell of claim 1, wherein said futile cycle of ATP consumption produces a flow of electrons through the respiratory machinery at a rate higher than endogenous electron flow of about 100-fold or more.

7. A microbial fuel cell, comprising a dissimilatory metal-reducing microbe expressing one or more exogenous genes encoding a gene product that promotes ATP consumption, said gene products of said one or more exogenous genes having an activity that reduces ATP synthesis, increases ATP consumption or both.

8. The microbial fuel cell of claim 7, wherein said one or more gene products increase ATP consumption through a futile cycle.

9. The microbial fuel cell of claim 7, wherein said one or more gene products increase ATP consumption through altering a metabolic reaction directly involved in ATP synthesis.

10. The microbial fuel cell of claim 7, wherein said one or more gene products increase ATP consumption through altering a metabolic reaction indirectly involved in ATP synthesis.

11. A microbial fuel cell, comprising a dissimilatory metal-reducing microbe expressing one or more exogenous genes encoding a gene products that increases the electron/mole ratio compared to an unmodified microbe, wherein the increased ratio enhances electron transfer to an electrode.

12. The microbial fuel cell of claim 11, wherein said one or more gene products comprise a glycerol processing operon.

13. The microbial fuel cell of claim 11, wherein said one or more gene products confers the ability of the microbe to use a substrate that is not possible to metabolize without the exogenous genes.

14. A method of producing electricity from an microbial organism, comprising:
(a) culturing a microbial fuel cell under anaerobic conditions sufficient for growth, said microbial fuel cell comprising a dissimilatory metal-reducing microbe expressing exogenous ATPase subunits, said ATPase subunits assembling into an active ATP synthase and consuming ATP in a futile cycle when grown under anaerobic conditions, and
(b) capturing electrons produced by an increased ATP demand with an electron acceptor.

15. The method of claim 14, wherein said dissimilatory metal-reducing microbe comprises an organism selected from the organisms set forth in Table 1.

16. The method of claim 14, wherein one or more exogenous ATPase subunits comprise a subunit selected from the ATPase subunits set forth in Tables 2 or 3.

17. The method of claim 14, wherein said futile cycle of ATP consumption produces a flow of electrons through the respiratory machinery at a rate higher than endogenous electron flow of about 2-fold, preferable about 5-fold, more preferably about 10-fold or more.

18. The method of claim 14, wherein said futile cycle of ATP consumption produces a flow of electrons through the respiratory machinery at a rate higher than endogenous electron flow of about 20-fold, preferably about 25-fold, more preferably about 50-fold or more.

19. The method of claim 14, wherein said futile cycle of ATP consumption produces a flow of electrons through the respiratory machinery at a rate higher than endogenous electron flow of about 100-fold or more.

20. The method of claim 14, wherein said electron acceptor comprises a graphite electrode.
